(19) United States
(12) Plant Patent Application Publication
(10) Pub. No.: US 2025/0212709 P1
(43) Pub. Date: Jun. 26, 2025

(54) Apple Tree Named 'CFS1'

(50) ;

(71) Applicant: The New Zealand Institute for Plant and Food Research Limited, Auckland (NZ)

(72) Inventor: Carl Fairey, Napier (NZ)

(73) Assignee: The New Zealand Institute for Plant and Food Research Limited, Auckland (NZ)

(21) Appl. No.: 18/934,018

(22) Filed: Oct. 31, 2024

Related U.S. Application Data
(60) Provisional application No. 63/601,667, filed on Nov. 21, 2023.

(51) Int. Cl. A01H 6/74; A01H 5/08

(52) U.S. Cl. CPC A01H 6/7418; A01H 5/08
USPC PLT/161

(57) ABSTRACT

A new and distinct variety of apple tree is described. The variety results from a limb sport found in Hawkes Bay, New Zealand. The fruit of the apple tree of this new variety have an attractive appearance, characterized by a high proportion of red over color and an over color patter that is a solid flush with weakly defined stripes.

### CROSS-REFERENCE TO RELATED APPLICATIONS

This application claims the benefit of U.S. Provisional Application No. 63/601,667, filed Nov. 21, 2023, the disclosure of which is incorporated by reference in its entirety.

Genus and species plant claimed: Malus domestica.

Variety denomination: ‘CFS1’.

### BACKGROUND OF THE INVENTION

The new variety of ‘CFS1’, Malus domestica, resulted from a spontaneous limb sport mutation of ‘Scifresh’ (U.S. Plant Pat. No. 13,888). The ‘Scifresh’ tree containing the limb sport of the new variety was discovered in 2012, growing in a cultivated area in Hawkes Bay, New Zealand. It was selected for further observation because of its fruit skin color. ‘CFS1’ was first asexually propagated in 2012 in Hawkes Bay, New Zealand, by budding onto rootstocks of ‘M9 337’ and these resulting trees were planted in an orchard in Hawkes Bay in 2014. Further propagation of the selection occurred in 2015, and more trees were planted in 2015 in Angers, France. The resulting plants were found to be true to type demonstrating that the characteristics of the new variety ‘CFS1’ are stable and transmitted without change through succeeding generations.

### SUMMARY OF THE INVENTION

‘CFS1’ can be characterized by its medium sized fruit (197 g) with red skin (near red, 46A) over color pattern that is a solid flush with weakly defined stripes.

‘CFS1’ is distinguished from its parent, ‘Scifresh’ by the following characteristics'

‘CFS1’ has on average a higher percentage of fruit skin over color (96%) compared to ‘Scifresh’ (73%). In addition, the red skin over color (near red, 46A) of ‘CFS1’ has a darker intensity compared to the over color of ‘Scifresh’, which is near 42D.

### BRIEF DESCRIPTION OF THE PHOTOGRAPHS

The accompanying photographs show typical specimens of the tree and fruit of the new variety as depicted in colors as nearly true as is reasonably possible to make the same in a color illustration of this character.

FIG. 1 shows ‘CFS1’ fruit on tree.

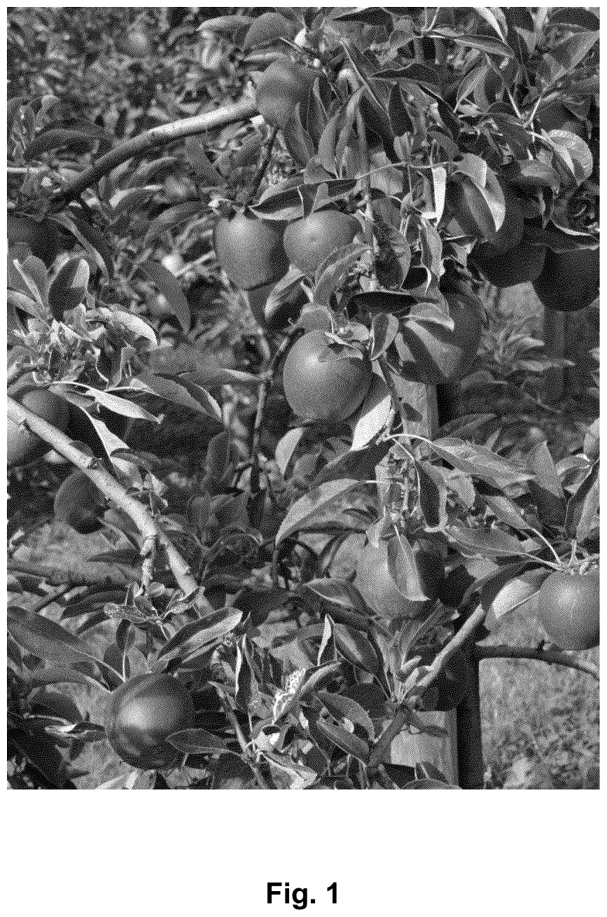

FIG. 2 shows ‘CFS1’ fruit in a lateral view.

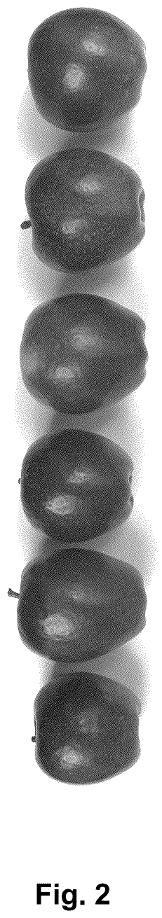

FIG. 3 shows ‘CFS1’ longitudinal sections of cut fruit.

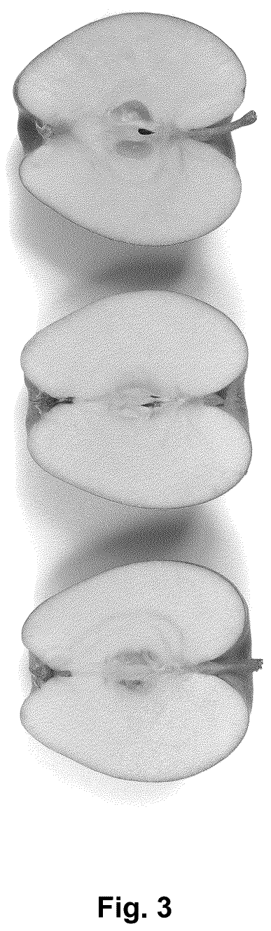

FIG. 4 shows a ‘CFS1’ 4-year-old tree.

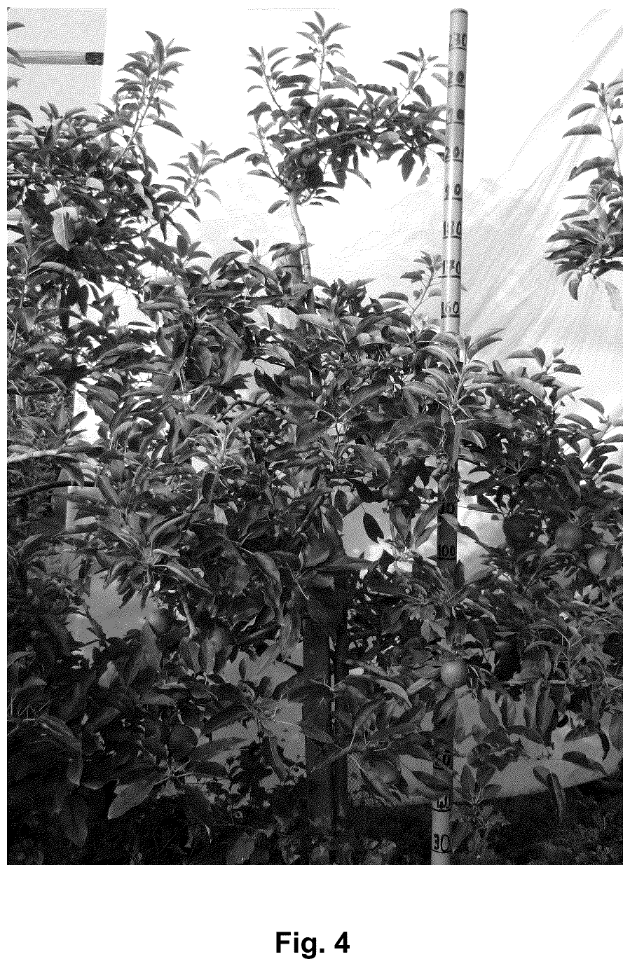

FIG. 5 shows the top side of ‘CFS1’ leaves.

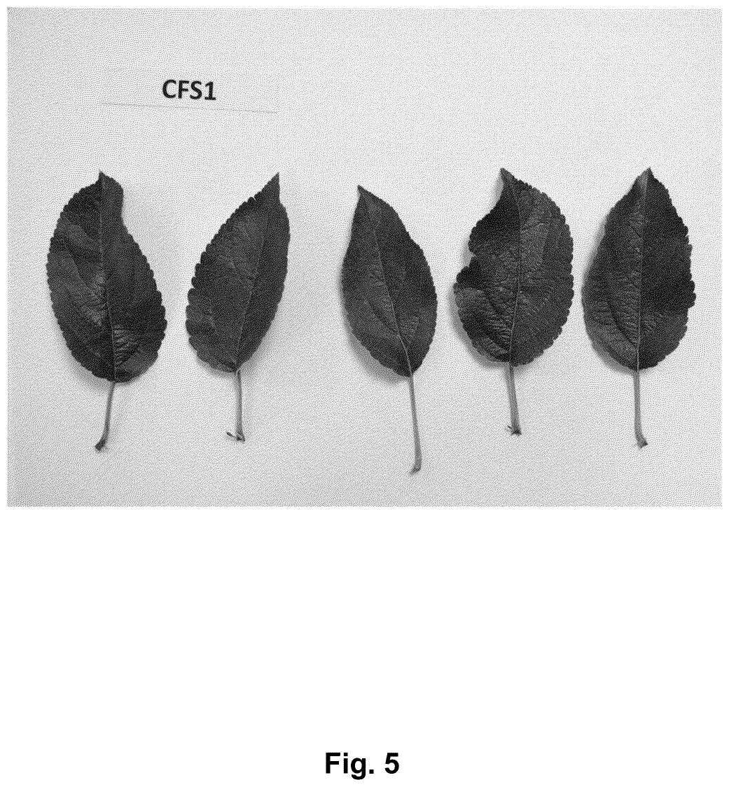

FIG. 6 shows ‘CFS1’ flowers.

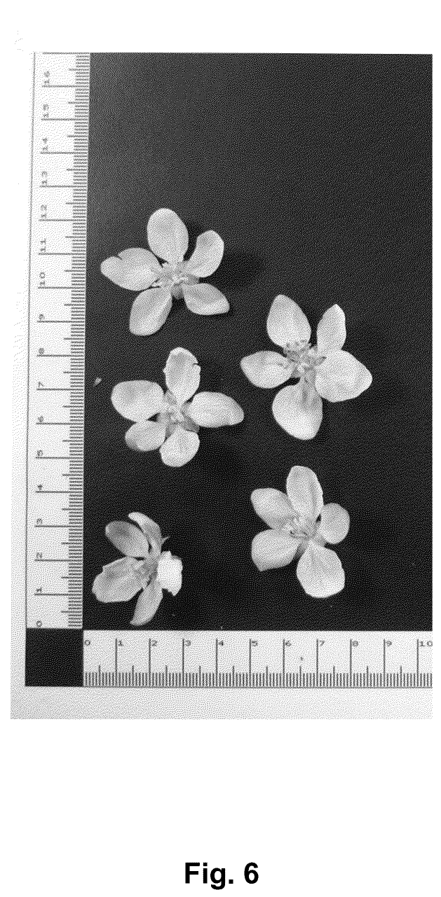

### DETAILED DESCRIPTION

The following is a detailed description of ‘CFS1’ with color terminology in accordance with The Royal Horticultural Society Color Charts (R.H.S.C.C) fifth edition.

The specimens described were grown at Angers, France. The observations were made over two seasons from 2019-2020 on 3 to 4-year-old trees on ‘M9’ rootstock. It should be understood that the characteristics described will vary somewhat depending on cultural practices and climate conditions and will also vary depending upon location and season. Quantified measurements are generally expressed as an average of measurements taken from individual trees of ‘CFS1’ unless otherwise noted. However, the measurements of any individual plant or any group of plants of ‘CFS1’ may vary from the stated average.
Characteristics of the variety—tree type: Ramified habit; approximately 2.37 m tall and 1.7 m wide; spreading habit; bearing on spurs and long shoots; medium vigor on ‘M9’, managed under standard orchard practice.
Trunk: Measurements were taken 20 cm from the graft union. Trunk circumference was approximately 10.34 cm; color near dark grey 197C; bark texture slightly rough.
One year old shoot: Approximately 22.3 cm long; medium thickness, averaging 5-6 mm with straight growth; color on the sunny side near medium brown, 200A; average internode length 1.99 cm; number of lenticels was medium, averaging 7.4/cm², elliptical to irregular round in shape and near greyed white in color, 156C; on the distal half of the shoot a medium amount of pubescence was present.
Vegetative bud: Approximately 5.7 mm long; acute shape and near brown 200A color; in relation to the shoot is adpressed to slightly held out.
Leaf blade: Average length 8.4 cm; average width 4.3 cm at the widest point; weak pubescence on the lower side; basal shape obtuse; apex shape acute; margin of the leaves crenate, the attitude in relation to the shoot outwards; color on upper side near dark green 147A; color on the underside near green 147C; color of the venation near light green 145C.
Petiole: Average length 30 mm; average width 3 mm; color on the upper side near green 139D and color on the underside near yellow green 145C.
Stipule: Average stipule length 3.925 mm; average width 0.9 mm; color on the upper side near yellow green 139C; under side color near green 139D.
Flowers: Predominate color at balloon stage near dark pink, near 53C; diameter of the open flower approximately 39.7 mm; an average of 4.7 flowers are present per cluster; location of flower bud mainly on spurs; fragrance present.
Petals: 5 petals per flower with a broad ovate shape; arrangement of petals intermediate; average length 17.15 mm; average width 10.9 mm; rounded base; lower surface color near red purple, 61C; near red purple veins, 71B; average length of the petal claw 2.04 mm.
Stamen: Average of 19.4 per flower; filaments average length 4.6 mm and near white in color, 155D; anthers average length 2.3 mm, color near yellow, 8C; pollen color near yellow, 7D.
Pistil: Average length 7.72 mm; color near light green, 145C; position of stigmas in relation to anthers: above.
Pedicel: Average length 17.6 mm; average diameter 1.19 mm; color near yellow green, 152B.
Sepals: Five sepals per flower; average length 7.55 mm and average width 3.55 mm; color near yellow green, 145A; attitude of sepals in relation to corolla recurved.
Fruit size: medium; average width 71.65 mm; average height 70.3 mm; average weight. 189 g.
Fruit shape: Most fruit tending to be conic in shape; position of maximum diameter middle; crowning at calyx absent to very weak; eye basin average depth 7.6 mm; average width 28 mm; ribbing absent; average length of sepals 5.1 mm.
Fruit skin: Bloom absent to very weak; greasiness absent to very weak; background color near yellow, 4A; size of lenticels small.
Fruit over color: Very large amount of over color, approximately 96%; near red, 46A, with medium to dark intensity; pattern of over color solid flush with weakly defined stripes; width of the stripes narrow; russet absent or very small amount around stalk attachment, on cheeks and around eye basin.

TABLE 1
Table showing the distinguishing features of 'CFS1' when compared to
'Scifresh. Observations made on trees in Angers, France over 2 seasons,
2019 and 2020, on trees aged 3-4 years, on 'M9' rootstock.

| Distinguishing | | |
|---|---|---|
| Characteristics | 'CFS1' | 'Scifresh' |
| Over color hue with | Near 53A | Near 42D |
| bloom removed | | |
| Relative area | Very large-on average | Medium on average |
| of over color | approximately 96% | approximately 73% |
| Ground color | Yellow, Near 4A | Yellow green, |
| of skin | | Near 154D |

Fruit stalk: Average length 14.6 mm; average width 3 mm; average depth of stalk cavity 10.5 mm; average width 34.2 mm; curvature of stalk absent or very weak.
Fruit Flesh: firm, average fruit firmness 9.2 kgf; near yellow white in color, 158A; aperture of locules in transverse section closed or slightly open.
Fruit flavor: Medium sweetness; medium acidity; aroma rich; average soluble solids 12.7%.
Fruit seeds: Five locules; ovate shaped; average length 9 mm; average width 5 mm; color near greyed orange, 175A.
Harvest: Late September.
Use: Dessert.
Pest and disease tolerance/susceptibility: ‘CFS1’ has not been observed to be any more or less tolerant or susceptible to any pest or diseases than ‘Scifresh’.

1. A new and distinct variety of apple tree named ‘CFS1’ substantially as illustrated and described herein.
